(19) United States
(12) Patent Application Publication
(10) Pub. No.: US 2024/0261356 A1
(43) Pub. Date: Aug. 8, 2024

(54) COMPOSITION CONTAINING ACANTHOPANAX EXTRACT AND GARCINIA CAMBOGIA EXTRACT OR COMPOUND ISOLATED THEREFROM AS ACTIVE INGREDIENT FOR PREVENTION OR TREATMENT OF LIVER DISEASE

(71) Applicant: KOREA RESEARCH INSTITUTE OF BIOSCIENCE AND BIOTECHNOLOGY, Daejeon (KR)

(72) Inventors: Su Ui LEE, Daejeon (KR); Mun-Ock KIM, Daejeon (KR); Hyung Won RYU, Daejeon (KR); Sei-Ryang OH, Daejeon (KR); Doo-Young KIM, Daejeon (KR); Jae-Won LEE, Daejeon (KR)

(73) Assignee: KOREA RESEARCH INSTITUTE OF BIOSCIENCE AND BIOTECHNOLOGY, Daejeon (KR)

(21) Appl. No.: 18/290,222

(22) PCT Filed: May 11, 2022
(86) PCT No.: PCT/KR2022/006785

§ 371 Date: Nov. 10, 2023

(30) Foreign Application Priority Data
May 12, 2021 (KR) 10-2021-0061558
May 4, 2022 (KR) 10-2022-0055720

(51) Int. Cl. A61K 36/254; A61K 31/191; A61K 31/365; A61K 36/38; A61P 1/16

(52) U.S. Cl. CPC A61K 36/254; A61K 31/191; A61K 31/365; A61K 36/38; A61P 1/16; A61K 2236/39

(57) ABSTRACT

The present invention relates to a composition containing extracts from Garcinia cambogia and Acanthopanax for prevention or treatment of liver diseases and, more specifically, to a composition for prevention or treatment of liver disease, the composition containing, as active ingredients, (i) a Garcinia cambogia extract, hydroxycitric acid (HCA), or a derivative of hydroxycitric acid; and (ii) an Acanthopanax extract, Chiisanogenin, or a derivative of Chiisanogenin.

### TECHNICAL FIELD

The present application claims priorities to Korean Patent Application No. 2021-0061558 filed on May 12, 2021 and Korean Patent Application No. 2022-0055720 filed on May 4, 2022, and the entire specification thereof is incorporated herein by reference.

The present invention relates to a composition for prevention or treatment of liver diseases, the composition comprising, as active ingredients, extracts from Acanthopanax and Garcinia cambogia or compounds isolated therefrom, and, more specifically, to a composition for prevention or treatment of liver diseases, the composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

### BACKGROUND ART

The liver has a variety of functions, in addition to important metabolic functions in internal organs, such as glycogen storage, red blood cell breakdown, plasma protein synthesis, and detoxification. Since the liver is the first defense organ that prevents damage from ingestion of foreign substances, a decline in liver function can cause serious diseases due to viruses or various drugs. Drugs that are toxic to the liver are also the main causes of liver diseases. Approximately half of severe liver diseases are caused by drugs, and most of them become chronic liver diseases.

Ethanol, the main ingredient in alcohol, has a wide and diverse range of physical and mental effects on the human body, and research has been conducted on its metabolic process and toxic properties. As a result of the metabolism of alcohol, a lot of fatty acids are produced and fat accumulates in the liver, which is called an ‘alcoholic fatty liver disease’. Such alcoholic fatty liver disease often progresses into a chronic disease, with alcoholic hepatitis occurring in 10 to 35% of cases and cirrhosis occurring in 8 to 20% of cases.

On the other hand, non-alcoholic liver damage refers to cases where a liver biopsy shows fatty change (steatosis) and lobular hepatitis (steatohepatitis), which are characteristic findings of alcoholic hepatitis, despite no history of alcohol intake that is recognized as harmful to the liver. The principle of treatment for non-alcoholic liver disease is improvement of lifestyle habits such as diet and exercise therapy, which is however difficult to be implemented reliably. Since nonalcoholic steatohepatitis has a high risk of progressing to liver cirrhosis or hepatocellular carcinoma, more active drug treatment is needed. Treatments aimed at improving oxidative stress and insulin resistance, which are considered important in the development and progression of non-alcoholic steatohepatitis, are being attempted. However, there is currently no treatment for which sufficient scientific evidence has been established.

Turmeric, whose main ingredient is curcumin, and Milk thistle, which belongs to the thistle family and contains silymarin as its main ingredient, as domestic liver function improvers, comprises antioxidant vitamins and flavonoids as main ingredients, and products made from dandelion root and Hovenia tree fruit, are being sold. In particular, the three functional raw materials related to improving liver function currently approved by the Food and Drug Administration are Hovenia chinensis, shiitake mycelium, and milk thistle, and most of the raw materials are dependent on imports.

### DISCLOSURE

### Technical Problem

Accordingly, the inventors of the present invention conducted repeated research to develop a new material that has excellent effects on various liver diseases and has fewer side effects. As a result, they discovered that the administration of a combination of Garcinia cambogia extract; and Acanthopanax extract or Chisanogenin produced a synergic effect of improving liver function, as compared to the administration of each substance alone, and have completed the present invention.

Therefore, an objective of the present invention is to provide a composition for prevention or treatment of liver diseases, the composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, an objective of the present invention is to provide a composition for prevention or treatment of liver diseases, the composition consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, an objective of the present invention is to provide a composition for prevention or treatment of liver diseases, the composition essentially consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

Another objective of the present invention is to provide a food composition for prevention or amelioration of liver diseases, the composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, another objective of the present invention is to provide a food composition for prevention or amelioration of liver diseases, the composition consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, another objective of the present invention is to provide a food composition for prevention or amelioration of liver diseases, the composition essentially consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

Another objective of the present invention is to provide a veterinary composition for prevention or amelioration of liver diseases, the composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, another objective of the present invention is to provide a veterinary composition for prevention or amelioration of liver diseases, the composition consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, another objective of the present invention is to provide a veterinary composition for prevention or amelioration of liver diseases, the composition essentially consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

Another objective of the present invention is to provide a feed composition for prevention or amelioration of liver diseases, the composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, another objective of the present invention is to provide a feed composition for prevention or amelioration of liver diseases, the composition consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, another objective of the present invention is to provide a feed composition for prevention or amelioration of liver diseases, the composition essentially consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

Another objective of the present invention is to provide use of (i) a Garcinia cambogia extract, hydroxycitric acid (HCA) or a derivative of hydroxycitric acid; and (ii) an Acanthopanax extract, Chiisanogenin or a derivative of Chiisanogenin, for the preparation of a composition for treating liver diseases.

Another objective of the present invention is to provide a method of treating liver diseases, the method comprising administering to a subject in need thereof an effective amount of a composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

### Technical Solution

In order to achieve the above-described objectives of the present invention, the present invention provides a composition for prevention or treatment of liver diseases, the composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, the present invention provides a composition for prevention or treatment of liver diseases, the composition consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, the present invention provides a composition for prevention or treatment of liver diseases, the composition essentially consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In order to achieve the another objective of the present invention, the present invention provides a food composition for prevention or amelioration of liver diseases, the composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, the present invention provides a food composition for prevention or amelioration of liver diseases, the composition consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, the present invention provides a food composition for prevention or amelioration of liver diseases, the composition essentially consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In order to achieve another objective of the present invention, the present invention provides a veterinary composition for prevention or amelioration of liver diseases, the composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, the present invention provides a veterinary composition for prevention or amelioration of liver diseases, the composition consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, the present invention provides a veterinary composition for prevention or amelioration of liver diseases, the composition essentially consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In order to achieve another objective of the present invention, the present invention provides a feed composition for prevention or amelioration of liver diseases, the composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, the present invention provides a feed composition for prevention or amelioration of liver diseases, the composition consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition, the present invention provides a feed composition for prevention or amelioration of liver diseases, the composition essentially consisting of, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In order to achieve another objective of the present invention, the present invention provides use of (i) a Garcinia cambogia extract, hydroxycitric acid (HCA) or a derivative of hydroxycitric acid; and (ii) an Acanthopanax extract, Chiisanogenin or a derivative of Chiisanogenin, for the preparation of a composition for treating liver diseases.

In order to achieve another objective of the present invention, the present invention provides a method of treating liver diseases, the method comprising administering to a subject in need thereof an effective amount of a composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

Hereinafter, the present invention will be described in detail.

The present invention provides a composition for prevention or treatment of liver diseases, the composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

Garcinia cambogia has been used in India and southern Asia for centuries as a souring agent for pork and fish and as a spice for curries. In Korea, a Garcinia cambogia extract is used as an auxiliary ingredient (which should be used in a minimum amount of 5% or less and daily intake that does not exceed 6 g) in formulas for weight control according to the ‘Food Code.’ In addition, in foreign countries such as the United States, Garcinia cambogia is sold as a dietary supplement or food ingredient at an intake level of 500 to 4,500 mg/day. Since there have been reports that consumption of Garcinia cambogia causes acute liver toxicity in some consumers, special caution is required regarding the dosage and administration cycle.

On the other hand, Acanthopanacis Cortex is a shrub plant belonging to the Araliaceae family and has five leaves. In Korea, there are fifteen (15) types of Acanthopanacis Cortex comprising Acanthopanax senticosus (RUPR. et MAX.) HARMS, Acanthopanax sessiliflorus (RUPR. et MAX.) SEEM.), and Acanthopanax senticosus var. subinermis KITAGAWA. Since it has no toxicity and side effects, Acanthopanacis Cortex has long been classified as an upper medicine in oriental medicine such that its roots and xylem (branches) have been used as medicine, and its leaves, fruits and flowers may also be used as medicinal parts. The leaves of Acanthopanacis Cortex contain chisanoside, and its roots contain not only Acanthopanacis Cortex glycosides Acanthoside B and D, but also sylrgin and coumarin glycosides. In addition, Acanthopanacis Cortex contains water-soluble polysaccharides that increase immunity. Acanthopanacis Cortex has a spicy, bitter, and warm taste and is known to act on the liver meridian and nerves to eliminate customs, boost energy, and call forth essence. In addition, it is known to relieve Oro (which refers to a disease caused by weakness of the five internal organs) and Chilsang (which refers to seven symptoms caused by weakness in men), be used for inability to use the legs, increase body energy, improve stamina, clear the mind, increase willpower, make the body lighter and prevent aging, purify bad blood in the body and treat various symptoms such as aching pain in the lumbar spine, male dysphoria, cystitis and female dysphoria.

In the present invention, the type of Acanthopanax is not particularly limited as long as it is a plant of the genus Acanthopanax. Non-limiting examples thereof may comprise Acanthopanax sessiliflorus (RUPR. et MAX.) SEEM., Acanthopanax senticosus (RUPR. et MAX.) HARMS, Acanthopanax senticosus var. subinermis KITAGAWA, Jiri Mountain Acanthopanax chiisanense NAKAI, Acanthopanax koreanum NAKAI, Acanthopanax rufinerve NAKAI, Acanthopanax seoulense NAKAI, Acanthopanax sieboloians and Acanthopanax sieboldianum MAKINO. In the present invention, the extract of Acanthopanax sessiliflorus (RUPR. et MAX.) SEEM may be one of the above-described plants of the genus Acanthopanax alone, or a mixed extract thereof, and, preferably, an extract of Acanthopanax sessiliflorus (RUPR. et MAX.) SEEM.

In one aspect of the present invention, the Garcinia extract and Acanthopanax extract may each independently be an extract from a flower, leaf, root, fruit, stem, root bark, stem bark or a combination thereof.

In the present invention, the Garcinia cambogia extract and the Acanthopanax extract obtained by extraction and separation from nature by using extraction and separation methods known in the art may be used. An “extract” as defined in the present invention is that extracted from Garcinia cambogia and/or Acanthopanax by using an appropriate solvent, and comprises, for example, crude extracts of Garcinia cambogia and/or Acanthopanax, polar solvent-soluble extracts, and non-polar solvent-soluble extracts.

Any pharmaceutically acceptable organic solvent may be used as an appropriate solvent for extracting the extract from the Garcinia cambogia and/or Acanthopanax, and water or an organic solvent may be used. Various solvents, e.g., purified water, alcohol (anhydrous or hydrous alcohol having 1 to 4 carbon atoms, comprising methanol, ethanol, propanol, isopropanol, butanol), acetone, ether, benzene, chloroform, ethyl acetate, methylene chloride, hexane and cyclohexane, although not limited thereto, may be used individually or in combination. Preferably, 10% (v/v) to 90% (v/v) of ethanol (alcohol), more specifically 10%, 20%, 30%, 40%, 50%, 60%, 70%, 80% or 90% ethanol (alcohol) may be used.

An extraction method may be selected from the group consisting of hot water extraction, cold needle extraction, reflux cooling extraction, solvent extraction, steam distillation, ultrasonic extraction, elution and compression. In addition, a conventional fractionation process may be performed on a desired extract, and the extract may be purified by using a conventional purification method. There is no limitation to the method for producing the Garcinia cambogia and/or Acanthopanax extract of the present invention, and any known method may be used.

For example, the Garcinia cambogia and/or Acanthopanax extract contained in the composition of the present invention may be prepared in a powder form by subjecting the primary extract extracted by the hot water extraction or solvent extraction method to additional processes such as reduced pressure distillation and freeze drying or spray drying. In addition, the primary extract may be further purified by using various chromatography methods such as silica gel column chromatography, thin layer chromatography and high performance liquid chromatography.

Therefore, in the present invention, Garcinia cambogia and/or Acanthopanax extract comprises all extracts, fractions and purifications, their dilutions, concentrates or dried products obtained at each stage of extraction, fractionation or purification.

In one aspect of the present invention, when the composition comprises Garcinia cambogia extract and Acanthopanax extract as active ingredients, the composition may be prepared by preparing Garcinia cambogia extract and Acanthopanax extract separately and then mixing them, or by mixing Garcinia cambogia and the Acanthopanax and then preparing the extract.

In the present invention, Chisanogenin is a compound having the structure of Formula 1 below:

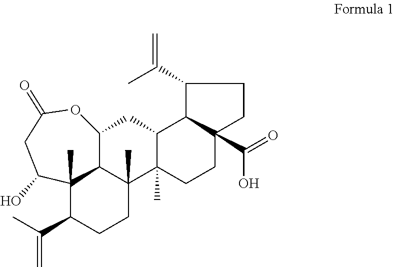

In the present invention, the Chiisanogenin may be isolated from a plant extract of the genus Acanthopanax or fractions thereof, purchased and used commercially, or prepared by a chemical synthesis method known in the art.

In one embodiment of the present invention, the Acanthopanax extract was subjected to chromatography by using a solvent with a ratio of hexane to acetone (a ratio of hexane to acetone of 4:1 to 1:1 (w/v)) to sequentially obtain four fractions, and the second fraction among them was subjected to chromatography by using a solvent with the ratio of methanol to water of 4:1 (w/v) to obtain two subfractions, and Chiisanogenin of formula 1 was isolated from the second fraction.

In the present invention, the Chiisanogenin comprises not only the compound of formula 1, but also a pharmaceutically acceptable salt thereof, and possible solvates, hydrates, racemates and stereoisomers that may be prepared therefrom.

The term “a derivative of Chiisanogenin” in the present invention refers to a compound or glycoside having the same core structure as chiisanogenin and comprises compounds that have an effect on improving liver diseases, but the present invention is not limited thereto. Examples thereof may comprise chiisanoside, 22-alpha-hydrochisanoside and 22-alpha-hydrochisanogenin.

According to one embodiment of the present invention, the administration of the combination of Garcinia cambogia extract and Acanthopanax extract, or Garcinia cambogia extract and Chiisanogenin was confirmed to have a synergic effect on improving the function of liver in a non-alcoholic fatty liver animal model, as compared to the single administration Specifically, the composition of the present invention was confirmed to of each substance. have the effects of inhibiting neutral fat accumulation in the liver tissue of a non-alcoholic fatty liver animal model, improving LDL levels in serum, and inhibiting FAS mRNA expression in a liver tissue. Considering that Garcinia cambogia extract may cause liver toxicity, the composition of the present invention may comprise Garcinia cambogia extract in combination with Acanthopanax extract or Chiisanogenin and administered with each substance at a dose that does not cause side effects or has a very low possibility of causing side effects, thereby achieving the desired effect of preventing or treating liver diseases.

In the present invention, the liver disease may be selected from the group consisting of non-alcoholic fatty liver disease, non-alcoholic steatohepatitis, alcoholic fatty liver disease, alcoholic liver damage, hepatitis, liver fibrosis, liver cirrhosis, liver failure, and liver cancer, and may preferably be liver fibrosis, liver cirrhosis, or liver failure caused by alcoholic liver damage, non-alcoholic fatty liver disease, non-alcoholic steatohepatitis, or non-alcoholic fatty liver disease.

In the present invention, the term “synergic effect” means that the effect that occurs when the components are administered in combination is greater than the sum of the effects that occur when each component is administered alone, as described in the literature.

The term “administered in combination” means that extracts or ingredients are administered to a subject together. That extracts or ingredients are administered together means that each ingredient may be administered sequentially at the same time or in any order or at different times to achieve a desired therapeutic effect.

It was confirmed that the use of the two ingredients in combination showed more excellent anti-fatty liver effects, as compared with the use of each ingredient alone. Examples of the present invention show that the combination of garcinia extract and Acanthopanax extract, or the combination of garcinia extract and Chiisanogenin may exhibit more excellent anti-fatty liver effects, as compared to the use of each ingredient alone. In particular, Garcinia extract and Acanthopanax extract mixed at a weight ratio of 1:1, or Garcinia extract and Chiisanogenin amixed at a weight ratio of 3:1 may exhibit more excellent anti-fatty liver effects, as compared to when mixed at any other ratio.

In one aspect of the present invention, (i) and (ii) may be contained in a weight ratio of 1:0.005 to 50.

Specifically, (i) and (ii) may be contained in a weight ratio of 1:0.005, 1:0.006, 1:0.007, 1:0.008, 1:0.009, 1:0.01, 1:0.02, 1:0.03, 1:0.04, 1:0.05, 1:0.06, 1:0.07, 1:0.08, 1:0.09, 1:0.1, 1:0.2, 1:0.3, 1:0.4, 1:0.5, 1:0.6, 1:0.7, 1:0.8, 1:0.9, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49 or 1:50.

In another aspect of the present invention, the (i) garcinia extract and the (ii) Acanthopanax extract may be characterized in that they are contained at a weight ratio of 1:0.005 to 25, preferably 1:0.05 to 5.0, more preferably 1:0.2 to 3, and most preferably 1:1 to 2.

In another aspect of the present invention, the (i) garcinia extract and the (ii) Chiisanogenin may be characterized in that they are contained at a weight ratio of 1:0.005 to 10, preferably 1:0.1 to 0.5, and most preferably 1:0.2 to 0.5.

In one aspect of the present invention, the (i) and (ii) contained in the composition of the present invention may be formulated in the form of a single composition or separate compositions and preferably formulated in the form of separate compositions. Techniques commonly used in the art may be used to formulate them.

In the pharmaceutical composition according to the present invention, the (i) and (ii) may be administered simultaneously, separately or sequentially. For example, if the components contained in the pharmaceutical composition of the present invention are in a single composition, they may be administered simultaneously, and, if the components are not in a single composition, they may be administered before, after, and/or simultaneously with the other components. The order of administration of the components contained in the pharmaceutical composition according to the present invention, i.e., at what point of time the components are to be administered simultaneously, separately or sequentially, may be determined by a doctor or expert. This order of administration may vary depending on many factors.

The pharmaceutical composition according to the present invention may comprise only (i) and (ii) above, or may be formulated in a suitable form comprising them together with a pharmaceutically acceptable carrier, and may additionally comprise excipients or diluents. The carrier comprises all types of solvents, dispersion media, oil-in-water or water-in-oil emulsions, aqueous compositions, liposomes, microbeads and microsomes.

Pharmaceutically acceptable carriers may further comprise, for example, carriers for oral administration or carriers for parenteral administration. Carriers for oral administration may comprise lactose, starch, cellulose derivatives, magnesium stearate, stearic acid, etc. In addition, the carriers for oral administration may contain various drug delivery substances used for oral administration. In addition, the carriers for parenteral administration may comprise water, suitable oil, saline solution, aqueous glucose, glycol, etc., and may further comprise stabilizers and preservatives. Suitable stabilizers comprise antioxidants such as sodium bisulfite, sodium sulfite or ascorbic acid. Suitable preservatives comprise benzalkonium chloride, methyl- or propyl-paraben and chlorobutanol. The pharmaceutical composition of the present invention may further comprise lubricants, wetting agents, sweeteners, flavoring agents, emulsifiers, suspending agents, etc., in addition to the above-described components. Other pharmaceutically acceptable carriers and formulations may refer to those known in the art.

In the present invention, the content of the composition is not greatly limited depending on the purpose or mode of use, for example, may be 0.01 to 99% by weight, preferably 0.5 to 50% by weight, and more preferably 1 to 30% by weight, based on the total weight of the composition. In addition, the pharmaceutical composition according to the present invention may further comprise additives such as pharmaceutically acceptable carriers, excipients or diluents, in addition to the active ingredients. The pharmaceutical composition of the present invention may comprise 0.1 to 99.9% by weight of a composition comprising (i) and (ii) prepared by the method of the present invention, and 99.9% to 0.1% by weight of a carrier.

The pharmaceutical composition according to the present invention may comprise a pharmaceutically effective amount of the compound alone or may additionally comprise one or more pharmaceutically acceptable carriers. The pharmaceutical composition of the present invention may be administered to a patient in a single dose or by a fractionated treatment protocol by which multiple doses are administered over a long period of time. In the above, the pharmaceutically effective amount refers to an amount that shows a greater response than the negative control and, preferably, to an amount sufficient to treat or prevent liver diseases. The effective amount of the composition according to the present invention may be 0.001 to 1000 mg/kg b.w./day, and preferably 1 to 2000 mg/kg b.w./day, but the present invention is not limited thereto. However, the pharmaceutically effective amount may vary appropriately depending on various factors such as a disease and its severity, patient's age, weight, health status and gender, an administration route and a treatment period.

The composition of the present invention may be administered to mammals, including human beings, by any method, for example, oral or parental administration. Parenteral administration methods may comprise, but are not limited to, intravenous, intramuscular, intraarterial, intramedullary, intrathecal, intracardiac, transdermal, subcutaneous, intraperitoneal, intranasal, enteral, topical, sublingual or rectal administration.

The pharmaceutical composition of the present invention may be formulated as a formulation for oral administration or parenteral administration according to the administration route described above.

In the case of the formulation for oral administration, the composition of the present invention may be formulated into powder, granules, tablets, pills, sugar-coated tablets, capsules, solutions, gels, syrups, slurries, suspensions, etc. by using methods known in the art. For example, the formulation for oral administration may be prepared by mixing an active ingredient with solid excipients, grinding the mixture, adding thereto suitable auxiliaries, and processing them into a granule mixture to obtain tablets or dragees. Examples of suitable excipients may comprise saccharides comprising lactose, dextrose, sucrose, sorbitol, mannitol, xylitol, erythritol and maltitol; starches comprising corn starch, wheat starch, rice starch and potato starch; celluloses comprising cellulose, methyl cellulose, sodium carboxymethylcellulose and hydroxypropylmethyl-cellulose; and fillers such as gelatin and polyvinylpyrrolidone. In addition, in some cases, cross-linked polyvinylpyrrolidone, agar, alginic acid or sodium alginate may be added as a disintegrant. Furthermore, the pharmaceutical composition of the present invention may further comprise anti-coagulants, lubricants, wetting agents, fragrances, emulsifiers and preservatives.

In the case of the formulation for parenteral administration, the composition of the present invention may be formulated in the form of injections, creams, lotions, ointments for external use, oils, moisturizers, gels, aerosols, and nasal inhalants by methods known in the art. These formulations are described in formulas generally known in all pharmaceutical chemistry.

The total effective amount of the composition of the present invention may be administered to a patient in a single dose, or may be administered by a fractionated treatment protocol in which multiple doses are administered over a long period of time. The dosage of ibuprofen and dexamethasone is determined by considering various factors such as formulation method, administration route, and number of treatments, as well as the patient's age, weight, health status, gender, severity of disease, diet, and excretion rate. Therefore, taking this into account, any one skilled in the art will be able to determine an appropriate effective dosage of the composition of the present invention.

On the other hand, when the (i) and (ii) are administered in the form of a single formulation, a formulation may be prepared by appropriately selecting the mixing ratio so that the daily dosage is at or below the maximum allowable value of each component, and the mixed formulation may be administered once a day or in several divided doses.

The pharmaceutical composition according to the present invention is not particularly limited in terms of its formulation, administration route, and administration method as long as it exhibits the effects of the present invention.

The mixing ratio, dosage, and administration time of the (i) and (ii) may be selected and administered by a person skilled in the art in consideration of the patient's pain level, patient's sensitivity to the drug, side effects, etc.

The pharmaceutical composition according to the present invention can exhibit an increased effect of preventing or treating increased liver diseases by administering the two components (i) and (ii) in combination, as compared to administration of each of them alone, and is effective in reducing side effects that may be caused by overdosing or long-term administration of each drug.

The present invention also provides a food composition for prevention or amelioration of liver diseases, the composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

The food composition of the present invention comprises all foods in the conventional sense, and comprises all forms such as functional foods, nutritional supplements, health foods, and food additives. The food compositions of such types may be prepared in various forms according to conventional methods known in the art.

The food composition of the present invention may comprise health functional foods. The term “health functional food” used in the present invention refers to food manufactured and processed in the form of tablets, capsules, powders, granules, liquids, and pills by using raw materials or ingredients with functional properties useful to the human body. Here, “functionality” means achieving useful effects for health purposes, such as regulating nutrients or performing physiological functions, in relation to the structure and function of the human body. The health functional food of the present invention may be manufactured by a method commonly used in the art, and may be manufactured by adding raw materials and components commonly employed in the art.

In addition, the formulation of the health functional food may be manufactured without limitation as long as it is a formulation recognized as a health functional food. The food composition of the present invention may be manufactured in a variety of formulations. In addition, unlike general drugs, it has food as a raw material and, thus, has the advantages of having no side effects that may be caused from taking a drug for a long period of time, and being highly portable. Therefore, the health functional food of the present invention can be consumed as an adjuvant to ameliorate liver diseases or enhance the treatment effect.

For example, the health functional food may be consumed in the form of tea, juice, and drinks comprising (i) and (ii) above, or may be consumed by granulation, encapsulation and powdering. In addition, it may be prepared in the form of a composition by mixing it with known substances or active ingredients known to have effects in preventing, improving or treating liver diseases.

In addition, the food composition of the present invention may contain various nutrients, vitamins, electrolytes, flavors, colorants, pectic acid and its salts, alginic acid and its salts, organic acids, protective colloidal thickeners, pH adjusters, stabilizers, preservatives, glycerin, alcohols, and carbonating agents used in carbonated beverages. The food composition may also contain pulp for the production of natural fruit juices, fruit juice drinks and vegetable drinks. Such ingredients may be used independently or in combination. Although the ratio of such additives is not very important, it is generally selected in the range of 0.01 to 0.3 parts by weight per 100 parts by weight of the food composition of the present invention. However, the present invention is not limited thereto.

In addition, the food composition of the present invention may comprise various flavoring agents or natural carbohydrates as additional components, like conventional beverages. The carbohydrates comprises monosaccharides such as glucose and fructose, disaccharides such as maltose and sucrose, polysaccharides such as dextrins and cyclodextrins, and sugar alcohols such as xylitol, sorbitol, and erythritol. Sweeteners may comprise natural sweeteners such as thaumatin and stevia extract, or synthetic sweeteners such as saccharin and aspartame. The ratio of the natural carbohydrate may be generally about 0.01 to 0.04 g, preferably about 0.02 to 0.03 g, per 100 mL of the composition of the present invention, but the present invention is not limited thereto.

The present invention also provides a veterinary composition for prevention or amelioration of liver diseases, the composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

The veterinary composition of the present invention may further comprise appropriate excipients and diluents according to conventional methods. The excipients and diluents may comprise lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, cetanol, stearyl alcohol, liquid paraffin, sorbitan monostearate, polysorbate 60, methylparaben, propylparaben and mineral oil.

The veterinary composition according to the present invention may further comprise fillers, anti-coagulants, lubricants, wetting agents, spices, emulsifiers, preservatives, etc. The veterinary composition according to the present invention may be formulated by using methods well known in the art to provide rapid, sustained or delayed release of the active ingredient after being administrated to an animal. The formulation may be in the form of powders, granules, tablets, capsules, suspensions, emulsions, solutions, syrups, aerosols, soft or hard gelatin capsules, suppositories, sterile injectable solutions, sterile external preparations, etc.

The veterinary composition according to the present invention may vary depending on the age, sex, and weight of an animal, but may be administered in an amount of 0.1 to 100 mg/kg once or several times a day. In addition, the dosage may be increased or decreased depending on the route of administration, severity of disease, gender, weight, age, etc. Therefore, the dosage does not limit the scope of the present invention in any way.

The present invention also provides a feed composition for prevention or amelioration of liver diseases, the composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

In addition to the composition according to the present invention, the “feed composition” may comprise food raw materials usable as foods as described in the Food Standards and Specifications (“Food Code”) and food additives described in the Food Additive Code as active ingredients, in addition to the above-described components according to the present invention. In addition, even if the feed composition does not comprise food raw materials or food additives usable as foods, it may comprise raw materials that fall within the scope of single feed in Annex 1 of “Standards and specifications for feed” and raw materials that fall within the scope of auxiliary feed in Annex 2.

The “feed composition” may be an extractant among supplementary feeds according to “Standards and specifications for feed,” or may be a compound feed comprising the supplementary feed.

When preparing the feed composition, the contents of (i) a Garcinia cambogia extract, hydroxycitric acid (HCA) or a derivative of hydroxycitric acid; and (ii) an Acanthopanax extract, Chiisanogenin or a derivative of Chiisanogenin do not need to be specifically limited as long as the feed composition shows prevention or amelioration of liver diseases, but may be, for example, 0.1 to 99% by weight, 0.5 to 95% by weight, It may be included in 1 to 90% by weight, 2 to 80% by weight, 3 to 70% by weight, 4 to 60% by weight and 5 to 50% by weight.

The content of the active ingredients (i) and (ii) in the feed composition may vary depending on the condition, body weight, and presence, degree, and period of disease of the ingesting animal, but may be appropriately selected by a person skilled in the art. For example, it may be 1 to 5,000 mg, preferably 5 to 2,000 mg, more preferably 10 to 1,000 mg, even more preferably 20 to 800 mg, and most preferably 50 to 500 mg, based on the daily dosage. The number of administrations does not need to be particularly limited, but may be adjusted by a person skilled in the art within the range of three times a day to once a week. In the case of long-term intake for health and hygiene purposes or health control, it may be below the above-described range.

The present invention also provides use of (i) a Garcinia cambogia extract, hydroxycitric acid (HCA) or a derivative of hydroxycitric acid; and (ii) an Acanthopanax extract, Chiisanogenin or a derivative of Chiisanogenin, for the preparation of a composition for treating liver diseases.

The present invention also provides a method of treating liver diseases, the method comprising administering to a subject in need thereof an effective amount of a composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

The term “effective amount” of the present invention refers to an amount that exhibits an effect of improving, treating, detecting, diagnosing of a wound, or inhibiting or reducing the progression of a wound, when administered to a subject. In addition, the term “individual” may be an animal, preferably a mammal, particularly an animal comprising a human being, and may also be a cell, tissue, or organ derived from an animal. The subject may be a patient in need of the effect.

The term “treatment” of the present invention comprehensively refers to improving wounded area or a symptom caused by a wound, may comprise curing, substantially preventing, or improving the condition of the wound, and comprises mitigating, curing, or preventing one or most of the symptoms caused by the disease, but the present invention is not limited thereto.

As used herein, the term “comprising” is used in the same sense as “including” or “characterized by.” The composition or method according to the present invention does not exclude additional components or method steps not specifically mentioned. In addition, the term “consisting of” refers to excluding additional elements, steps or components not separately described. The term “essentially consisting of” means that, in addition to the described materials or steps, materials or steps that do not substantially affect the basic characteristics thereof may be contained in the scope of a composition or method.

### Advantageous Effects

The composition provided by the present invention can exhibit an increased effect of presenting or treating liver diseases by administering a Garcinia cambogia extract; and an Acanthopanax extract or Chiisanogenin in combination, as compared to administration of each substance alone, and be effective in reducing the possibility of any side effects that may be caused from excessive or long-term administration of each substance.

### DESCRIPTION OF DRAWINGS

FIG. 1 is a result showing the yield of Chiisanogenin according to the conditions (e.g., solvent and temperature) for extracting Acanthopanax.

FIG. 2 is a schematic diagram of the fractionation steps for obtaining Chiisanogenin from an Acanthopanax extract.

### MODE FOR INVENTION

Hereinafter, the present invention will be described in detail by the following embodiments. However, the following embodiments are only for illustrating the present invention, and the present invention is not limited thereto.

### Example 1: Preparation of Experimental Materials

(1) Preparation of a Garcinia cambogia Extract

The raw material distributed by Sabinsa Korea Corporation (Batch No. C150292E) was used as a Garcinia cambogia extract, and HCA was purchased from Chromadex (ASB-00008387-250).

### (2) Preparation of an Acanthopanax Extract

3 g of crushed Acanthopanax leaves (Scientific name: Eleutherococcus sessiliflorus (Rupr. & Maxim.); Voucher number: KRIB 0079085-0079086; Where to buy: Susin-myeon, Cheonan-si, Chungcheongnam-do, Korea) was put into a bottle, 100 mL of a solvent under the above-described conditions for solvent was added, and the mixture was extracted for 5 hours in a water bath under the following temperature conditions, followed by cooling at room temperature for one hour. The mixture was filtered on filter paper (advantec NO. 2, 5 μm) and then concentrated, 30 mL of 50% (samples at 30° ° C. and 60° C.) and 100% MeOH (a sample at 90° C.) solvents were precisely measured to completely dissolve the extracts, followed by repetition of the extraction once by using the same method, filtering through a syringe filter (advantec, 0.2 μm) and analysis by UPLC. The obtained extract (30 mg/mL) was used to perform response surface analysis.

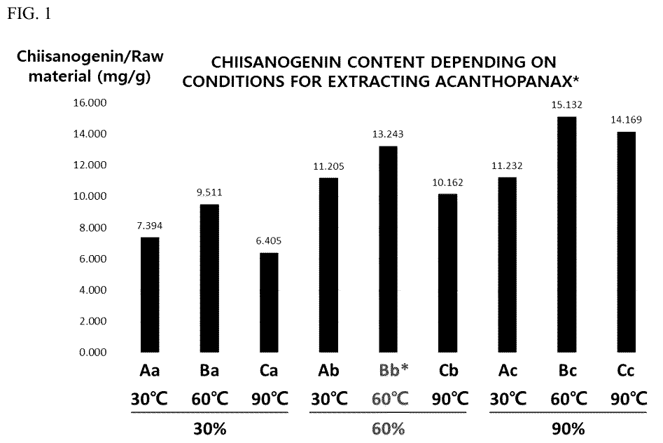

Nine (9) types of extracts were finally obtained by applying various ethanol concentrations (i.e., 30%, 60% and 90%) and extraction temperatures (i.e., 30° C., 60° C. and 90° C.), and the Chiisanogenin contents in the dried raw materials were measured and are shown in FIG. 1.

Referring to FIG. 1, it is confirmed that the contents of Chiisanogenin in the raw materials appear high under certain extraction conditions. In particular, it was confirmed that the extraction temperature of 60° C. maximized the Chiisanogenin content in the ethanol solvents having all concentrations. In addition, as a result of statistical analysis using response surface analysis by using minitab 18 (Eretec Inc), it was confirmed that 60% ethanol was the optimal condition at 60° C.

### (3) Isolation of Chiisanogenin

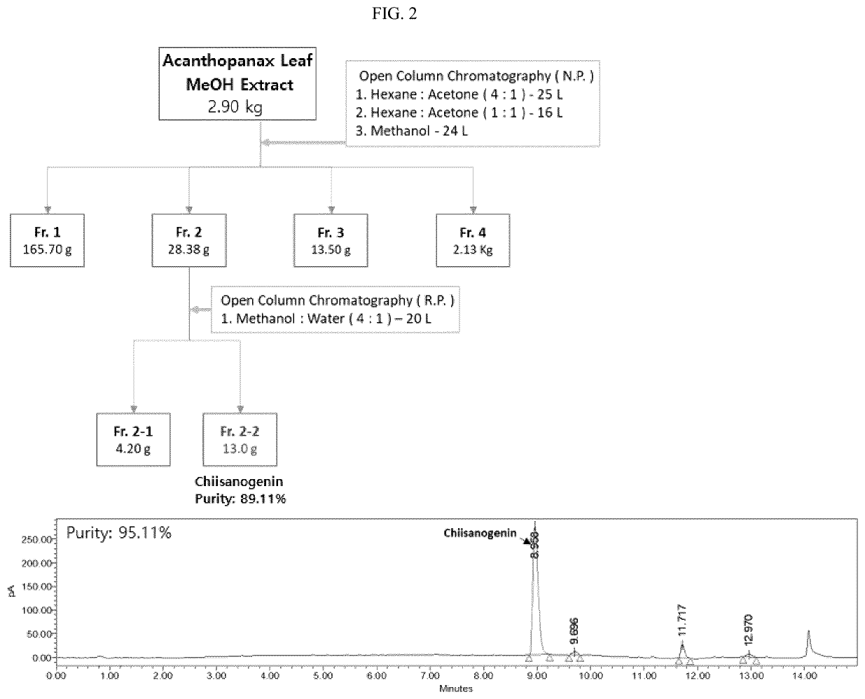

15.0 kg of Acanthopanax leaves were subjected to solvent extraction at a temperature of 30° C. by using 80% methanol, which was repeated three times for 3 days. 2.9 kg of the extract was subjected to open column chromatography (N.P.) to obtain four fractions (i.e., Fr1 to Fr4). The solvent used at this time had a ratio of hexane to acetone of 4:1 to 1:1 (W/V). Among the four fractions, Fr.2 (28.38 g) was subjected to open column chromatography by using a solvent with the ratio of methanol to water of 4:1 (W/V) to obtain two small fractions (i.e., Fr.2-1 and Fr.2-2), thereby obtaining Compound 1 (Fr.2-2, Chiisanogenin, 13.0 g) represented by formula 1 in a lignan structure having the following physical properties (FIG. 2).

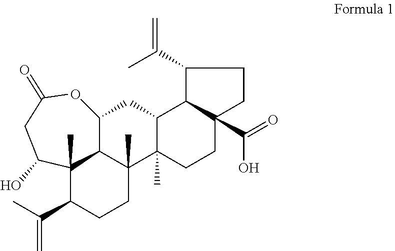

The components of the extract and fractions were analyzed by using ACQUITY™ Ultra Performance Liquid Chromatography (UPLC) and Charged Aerosol Detector (CAD) detectors. At this time, 0.1% FA (Formic acid)/DW was used as solvent A, and 0.1% FA/ACN (acetonitrile) solvent was used as solvent B. The analysis was performed under the gradient conditions shown in Table 1 below and the CAD conditions shown in Table 2 below.

TABLE 1

| Time (min) | Flow (ml/min) | % A | % B |
|---|---|---|---|
| 0.00 | 0.400 | 90 | 10 |
| 1.20 | 0.400 | 90 | 10 |
| 3.00 | 0.400 | 70 | 30 |
| 4.00 | 0.400 | 65 | 35 |
| 9.00 | 0.400 | 30 | 70 |
| 11.00 | 0.400 | 0 | 100 |
| 13.10 | 0.400 | 0 | 100 |
| 13.20 | 0.400 | 90 | 10 |
| 15.00 | 0.400 | 90 | 10 |

TABLE 2

| | Conditions |
|---|---|
| Filter | 2.0 |
| Data Rate | 20 Hz |

ACQUITY UPLC® BEH C18, 1.7 μm, 2.1×100 mm was used as the column. 5 μl of the extract was used at a concentration of 1 mg/ml.

### Example 2: Evaluation of Liver Function Improvement Efficacy

### (1) Experimental Method

### 1) Non-Alcoholic Fatty Liver Animal Models

### 1-1) Method of Treating Samples

8-week-old male C57bl/6J mice were adapted to basic feed (AlN-76A diet) for 2 weeks and then fed a HFD diet with 60 kcal % Diet Induced Obesity Diet Formulas (D12451) for 2 weeks from 10 weeks of age. When their average body weights reached at least 28 g, single samples (i.e., Garcinia extract, Acanthopanax extract or Chiisanogenin) and a composite sample thereof were administered for 10 weeks, together with the HFD diet.

### 1-2) Analysis of Non-Alcoholic Fatty Liver Animal Model

It was analyzed whether the triglyceride content accumulated in the liver due to high-fat diet intake was inhibited by administration of the composite composition of the Garcinia extract, Acanthopanax extract and Chiisanogenin, as compared to administration of single compositions. To measure the fat content deposited in the liver, 0.1 g of liver was homogenized, and the triglyceride (TG) content in the supernatant was measured by using an automatic biochemical analyzer (Hitachi-720, Hitachi Medical, Japan). The results are shown in Table 1 below. To measure low-density lipoprotein (LDL) in serum, plasma isolated from experimental animals was measured by using an automatic biochemical analyzer. The results are shown in Table 2 below. RNA was extracted by using RNAsolB (Tel-Test) solution, followed by cDNA synthesis and real-time PCR analysis by using one-step SYBR Green PCR kit (AB science) to determine the fatty acid synthase (FAS) gene expression pattern in the liver extracted from each experimental animal. As primers for PCR, Forward 5′-CTGAGATCCCAGCACTTCTTGA-3′ (SEQ ID NO: 1) and Reverse 5′-GCCTCCGAAGCCAAATGAG-3′ (SEQ ID NO: 2) were used. Real time quantitative PCR was performed by using the Applied Biosystems 7500 Real-Time PCR system (Applied Biosystems, USA). The results are shown in Table 3 below.

### 2) Non-Alcoholic Steatohepatitis (NASH) Animal Model

### 2-1) Method of Treating Samples

8-week-old male C57bl/6J mice were fed a NASH-inducing diet (#A06071302 diet) for 8 weeks, and, from the 9th week, single samples (i.e., Garcinia extract, Acanthopanax extract or Chiisanogenin) and a composite sample thereof were administered for 8 weeks, together with NASH dietary feed (#A06071302 diet).

### 2-2) Analysis of Non-Alcoholic Steatohepatitis Animal Model

To verify the efficacy of each of the Garcinia extract (notified raw material, 60% HCA) and Chiisanogenin or their mixture at each non-clinical administration concentration by using a non-alcoholic steatohepatitis (NASH)-induced animal model, changes of aspartate aminotransferase (AST) and alanine aminotransferase (ALT), which are biomarkers of liver tissue damage, in the serum of the non-alcoholic steatohepatitis (NASH) C57bl/6J mouse models caused by feeding NASH-inducing dietary feed (#A06071302) were observed, and the results are shown in Tables 11 and 12, respectively. In addition, the results of observing changes of hydroxyproline, which is a biomarker of liver fibrosis, in liver tissues are shown in Table 13.

### 3) Experiments of Fatty Acid-Induced Intracellular Fat Accumulation

The human liver-derived HepG2 cell line was purchased from American Type Culture Collection (ATCC, VA, USA). HepG2 cells were cultured at 37° C. in an incubator supplied with 5% carbon dioxide. For the cell culture, MEM (Gibco Cat No. 11095-098) medium containing 10% fetal bovine serum (FBS) and 1% antibiotics (Penicillin-streptomycin, Gibco) was used. HepG2 cells were seeded in a 96-well plate at 1×10⁴ cells/well, and, after 24 hours, treated with 150 μM oleic acid (Sodium oleate, Sigma, USA) for 24 hours to increase intracellular fat accumulation. The drug treated group was treated 1 hour before being treated with oleic acid. Upon completion of the experiment, the cells were fixed with 4% formaldehyde for 10 minutes and washed twice with PBS buffer. A solution containing BODIPY 490/503 (Invitrogen Corporation, NY, USA) was added and cultured at room temperature for 30 minutes. At this time, the final concentration of the fluorescent dye BODIPY was 2 μg/ml. After completion of the reaction, the plate was washed three times with PBS buffer, and, when only the minimum amount of PBS remained, the fluorescence was quantified by using a SpectraMax M4 microplate reader (Molecular Devices, USA). At this time, the excitation wavelength was 485 nm, and the emission was 525 nm. The experiment was performed by blocking light with aluminum foil to allow in as little light as possible. The results are shown in Tables 6 and 7 below.

In the experimental conditions of the present invention, cell viability was determined by mixing 90 μl of medium with 10 μl of CCK-8 solution, adding 100 μl per well and reacting the mixture for at least 30 minutes to 1 hour, followed by measurement of the absorbance at 450 nm, as described in the CCK-8 (Dojindo) kit. The cell viability was calculated according to Equation 1 below, taking the negative control as 100%, and the results are shown in Tables 8 and 9 below.

$$Cell viability ( \% ) = ( OD 450 nm value of extract - treated \\ group / OD 450 nm value of negative control group ) \times 100 \qquad Equation 1$$

### 4) Analysis of Alcoholic Liver Damage Recovery Efficacy

To culture HepG2 cells, which are a human-derived liver cancer cell line, they were suspended at a concentration of 1× 10⁶ cells/well by using DMEM medium (WEKGEBE, Korea) supplemented with 10% fetal bovine serum, and cultured in a 60 mm dish (Thermo Fisher Scientific), and the cells were cultured for 24 hours. To induce alcoholic liver damage after 24 hours, DMEM medium containing 10% FBS was exchanged for the medium containing 0.4M ethyl alcohol (Sigma aldrich), and the cells were cultured for 24 hours. To confirm the effect of the drug during alcoholic liver damage, the medium was treated with the drug during the exchange. To confirm liver damage recovery, 5 mg/ml MTT Solution (Thiazolyl Blue Tetrazolium Bromide, Sigma Aldrich) was added to each dish to a concentration of 100 mg/ml and incubated for 30 minutes. Afterwards, the MTT solution was removed, the resulting fomazan was dissolved in 2 ml DMSO, 100 μl each was transferred to a 96-well plate, and the absorbance was measured at 540 nm. The cell growth rate was calculated according to Equation 1 above, and the results are shown in Table 10 below.

### (2) Experimental Results

The results of each experiment performed according to the above-described experimental method are shown in Tables 3 to 9 below.

TABLE 3
Inhibitory effect of intracellular fat accumulation in fatty acid-
induced non-alcoholic fatty liver cell models, depending on a mixing
ratio of a Garcinia extract and an Acanthopanax extract

| | | Treatment concentration | | TG (% relative | |
|---|---|---|---|---|---|
| | | (μg/ml) | | to positive | |
| | Fatty | Garcinia | Acanthopanax | control group, | Inhibition |
| No. | acid | extract | extract | mean ± deviation) | rate (%) |
| 1 | − | 0 | 0 | 64 ± 9.5 | — |
| 2 | + | 0 | 0 | 100 ± 0.0 ## | — |
| 3 | + | 150 | 0 | 95 ± 14.5 | 14 |
| 4 | + | 135 | 2.5 | 93 ± 15.1 | 21 |
| 5 | + | 120 | 5 | 91 ± 1.0 | 25 |
| 6 | + | 105 | 7.5 | 85 ± 8.7 | 38 |
| 7 | + | 90 | 10 | 76 ± 11.5 | 35 |
| 8 | + | 60 | 15 | 77 ± 4.5 ** | 53 |
| 9 | + | 45 | 17.5 | 82 ± 5.9 ** | 51 |
| 10 | + | 30 | 20 | 80 ± 5.7 ** | 56 |
| 11 | + | 15 | 22.5 | 73 ± 7.5 ** | 85 |
| 12 | + | 0 | 25 | 89 ± 14.9 | 31 |

As shown in Table 3 above, it was confirmed that a synergistic effect was observed when the Garcinia extract and Acanthopanax extract were treated in combination, as compared to when they were treated individually. In particular, it was confirmed that the combined use thereof showed the best effect when the weight ratio of Garcinia extract to Acanthopanax extract was 60:15 to 15:22.5.

TABLE 4
Inhibitory effect of intracellular fat accumulation in fatty
acid-induced non-alcoholic fatty liver cell models, depending
on a mixing ratio of a Garcinia extract and Chiisanogenin

| | | Treatment concentration | | TG (% relative | |
|---|---|---|---|---|---|
| | | (μg/ml) | | to positive | |
| | Fatty | Garcinia | Acanthopanax | control group, | Inhibition |
| No. | acid | extract | extract | mean ± deviation) | rate (%) |
| 1 | − | 0 | 0 | 55 ± 4.2 | — |
| 2 | + | 0 | 0 | 100 ± 11.1 ### | — |
| 3 | + | 150 | 0 | 86 ± 5.3 * | 32 |
| 4 | + | 135 | 0.6 | 84 ± 12.6 | 37 |
| 5 | + | 120 | 1.2 | 76 ± 2.9 ** | 53 |
| 6 | + | 105 | 1.8 | 73 ± 3.6 ** | 61 |
| 7 | + | 90 | 2.4 | 76 ± 1.6 * | 53 |
| 8 | + | 75 | 3 | 75 ± 7.2 * | 56 |
| 9 | + | 60 | 3.6 | 70 ± 4.6 ** | 67 |
| 10 | + | 45 | 4.2 | 71 ± 6.7 ** | 64 |
| 11 | + | 30 | 4.8 | 60 ± 6.4 ** | 89 |
| 12 | + | 15 | 5.4 | 52 ± 7.3 *** | 109 |
| 13 | + | 0 | 6 | 64 ± 7.9 ** | 80 |

As shown in Table 4 above, it was confirmed that a synergistic effect was observed when the Garcinia extract and Chiisanogenin were treated in combination, as compared to when they were treated individually. In particular, it was confirmed that the combined use thereof showed the best effect when the weight ratio of Garcinia extract to Chiisanogenin was 30:4.8 to 15:5.4.

TABLE 5
Changes in cell viability of fatty acid-induced non-alcoholic
fatty liver cell models by treatment of each of a Garcinia
extract (notified raw material, 60% HCA) and an Acanthopanax
extract and combinations thereof

| | |
|---|---|
| Treatment concentration | |
| (μg/ml) | Cell viability |

| | Fatty | Garcinia | Acanthopanax | (%, mean ± |
|---|---|---|---|---|
| No. | acid | extract | extract | deviation) |
| 1 | − | 0 | 0 | 100 ± 3.7 |
| 2 | + | 0 | 0 | 113 ± 10.2 |
| 3 | + | 150 | 0 | 112 ± 10.8 |
| 4 | + | 135 | 2.5 | 116 ± 13.6 |
| 5 | + | 120 | 5 | 154 ± 19.9 |
| 6 | + | 105 | 7.5 | 147 ± 16.9 |
| 7 | + | 90 | 10 | 127 ± 19.4 |
| 8 | + | 60 | 15 | 112 ± 3.9 |
| 9 | + | 45 | 17.5 | 115 ± 8.4 |
| 10 | + | 30 | 20 | 125 ± 7.5 |
| 11 | + | 15 | 22.5 | 119 ± 5.6 |
| 12 | + | 0 | 25 | 139 ± 15.1 |

TABLE 6
Changes in cell viability of fatty acid-induced non-
alcoholic fatty liver cell models by treatment of each
of a Garcinia extract (notified raw material, 60%
HCA) and Chiisanogenin and combinations thereof

| | |
|---|---|
| Treatment concentration | |
| (μg/ml) | Cell viability |

| | Fatty | Garcinia | Acanthopanax | (%, mean ± |
|---|---|---|---|---|
| No. | acid | extract | extract | deviation) |
| 1 | − | 0 | 0 | 100 ± 2.4 |
| 2 | + | 0 | 0 | 141 ± 8.7 |
| 3 | + | 150 | 0 | 144 ± 11.4 |
| 4 | + | 135 | 0.6 | 141 ± 8.8 |
| 5 | + | 120 | 1.2 | 143 ± 5.4 |
| 6 | + | 105 | 1.8 | 142 ± 7.4 |
| 7 | + | 90 | 2.4 | 138 ± 22.4 |
| 8 | + | 75 | 3 | 136 ± 16.4 |
| 9 | + | 60 | 3.6 | 119 ± 12.1 |
| 10 | + | 45 | 4.2 | 123 ± 15.6 |
| 11 | + | 30 | 4.8 | 115 ± 12.2 |
| 12 | + | 15 | 5.4 | 117 ± 12.0 |
| 13 | + | 0 | 6 | 105 ± 13.7 |

Meanwhile, Tables 5 and 6 are results showing cell viability under the same conditions as those of the experiments in Tables 3 and 4, respectively, and it was confirmed that there was no significant change in cell viability caused from treatment with drugs under the conditions of this experiment.

Based on the cell experiment results, experimental groups were formed with the content ratio that showed the best combination effect, and the following in vivo efficacy evaluation experiment was conducted.

TABLE 7
Inhibition rate of neutral fat accumulation in the liver
tissues of non-alcoholic fatty liver animal models by a
Garcinia extract, an Acanthopanax extract and Chiisanogenin

| | | | TG (% relative to | |
|---|---|---|---|---|
| | | Concen- | positive control | Inhibition rate |
| | | tration | group, mean ± | (%, mean ± |
| No. | Sample | (mg/kg) | deviation) | deviation) |
| 1 | Normal | 0 | 19 ± 11.0 | — |
| 2 | HFD | 0 | 100 ± 0.8 ## | — |
| 3 | Garcinia extract | 200 | 99 ± 5.3 | 2 ± 6.2 |
| 4 | Acanthopanax | 200 | 71 ± 16.6 | 35 ± 17.2 |
| | extract | | | |
| 5 | Garcinia extract | 100 | 59 ± 11.8 * | 50 ± 8.2 |
| | Acanthopanax | 100 | | |
| | extract | | | |
| 6 | Garcinia extract | 200 | 30 ± 3.2 ** | 87 ± 9.0 |
| | Acanthopanax | 200 | | |
| | extract | | | |
| 7 | Garcinia extract | 150 | 36 ± 0.5 *** | 78 ± 11.6 |
| | Chiisanogenin | 50 | | |
| 8 | Garcinia extract | 300 | 30 ± 3.7 ** | 87 ± 8.3 |
| | Chiisanogenin | 100 | | |

As shown in Table 7 above, it was found that the effect of reducing the triglyceride content accumulated in the liver due to high-fat diet intake was higher in the groups administered with a composite composition of the Garcinia extract, Acanthopanax extract and Chiisanogenin, as compared to the groups administered with each of them alone.

TABLE 8
Changes of LDL in the serum of non-alcoholic fatty
liver animal models by a Garcinia extract, an Acanthopanax
extract and a Chiisanogenin compound

| | | | LDL in serum | |
|---|---|---|---|---|
| | | | (% relative to | |
| | | Concen- | positive control | Inhibition rate |
| | | tration | group, mean ± | (%, mean ± |
| No. | Sample | (mg/kg) | deviation) | deviation) |
| 1 | Normal | 0 | 59 ± 11.4 | — |
| 2 | HFD | 0 | 100 ± 19.9 ## | — |
| 3 | Garcinia extract | 200 | 89 ± 22.8 | 26 ± 10.2 |
| 4 | Acanthopanax | 200 | 83 ± 9.7 * | 40 ± 13.6 |
| | extract | | | |
| 5 | Garcinia extract | 100 | 79 ± 12.3 * | 51 ± 6.6 |
| | Acanthopanax | 100 | | |
| | extract | | | |
| 6 | Garcinia extract | 150 | 79 ± 12.5 * | 52 ± 6.0 |
| | Chiisanogenin | 50 | | |

As shown in Table 8 above, it was confirmed that the effect of reducing the LDL level in serum increased by HFD administration was significantly higher in the groups administered with a composite composition of the Garcinia extract. Acanthopanax extract and Chiisanogenin, as compared to the groups administered with each of them alone.

TABLE 9
Inhibitory effect of FAS mRNA expression in liver tissues of
non-alcoholic fatty liver animal models by a Garcinia extract,
an Acanthopanax extract and a Chiisanogenin compound

| | | | FAS mRNA | |
|---|---|---|---|---|
| | | | expression (% | |
| | | | relative to | |
| | | Concen- | positive control | Inhibition rate |
| | | tration | group, mean ± | (%, mean ± |
| No. | Sample | (mg/kg) | deviation) | deviation) |
| 1 | Normal | 0 | 11 ± 5.4 | — |
| 2 | HFD | 0 | 100 ± 30.2 ### | — |
| 3 | Garcinia extract | 200 | 82 ± 10.7 | 20 ± 14.5 |
| 4 | Acanthopanax | 200 | 78 ± 17.1 | 24 ± 8.1 |
| | extract | | | |
| 5 | Garcinia extract | 100 | 42 ± 34.0 ** | 65 ± 16.1 |
| | Acanthopanax | 100 | | |
| | extract | | | |
| 6 | Garcinia extract | 150 | 33 ± 17.4 *** | 75 ± 4.2 |
| | Chiisanogenin | 50 | | |

As shown in Table 9 above, it was confirmed that the effect of reducing gene expression of FAS in livers extracted from the experimental animals was significantly higher in the groups administered with a composite composition of the Garcinia extract, Acanthopanax extract and Chiisanogenin, as compared to the groups administered with each of them alone.

TABLE 10
Alcoholic liver damage recovery cell experiment

| | | Treatment concentration | | |
|---|---|---|---|---|
| | | (μg/ml) | | |
| | EtOH | Garcinia | Acanthopanax | Cell viability |
| No. | (0.5M) | extract | extract | (%, mean ± deviation) |
| 1 | − | 0 | 0 | 100 ± 2.6 |
| 2 | + | 0 | 0 | 9 ± 0.0 ### |
| 3 | + | 40 | 0 | 9 ± 0.1 |
| 4 | + | 0 | 40 | 11 ± 0.1 *** |
| 5 | + | 40 | 40 | 14 ± 0.2 *** |
| 6 | + | 80 | 80 | 27 ± 0.7 *** |

As shown in Table 10 above, it was confirmed that the inhibitory effect of cell death by alcohol, i.e., liver cell protection effect, more increased in the groups administered with a composite composition of the Garcinia extract and Acanthopanax extract, as compared to the groups administered with each of them alone.

TABLE 11
Changes of AST in the serum of nonalcoholic
steatohepatitis (NASH) animal models

| | | | AST in serum (% | |
|---|---|---|---|---|
| | | | relative to | |
| | | Concen- | positive control | Inhibition rate |
| | | tration | group, mean ± | (%, mean ± |
| No. | Sample | (mg/kg) | deviation) | deviation) |
| 1 | Control material | 0 | 30 ± 4.9 | — |
| | (normal animal) | | | |
| 2 | Control material | 0 | 100 ± 26.6 ### | — |
| | (NASH animal) | | | |
| 3 | Garcinia extract | 200 | 85 ± 14.3 | 21 ± 0.5 |
| 5 | Chiisanogenin | 50 | 97 ± 15.4 | 4 ± 19.8 |
| 6 | Garcinia extract | 75 | 72 ± 10.6 ** | 40 ± 24.1 |
| | Chiisanogenin | 25 | | |
| 7 | Garcinia extract | 150 | 65 ± 15.4 ** | 50 ± 26.1 |
| | Chiisanogenin | 50 | | |

AST is an enzyme present in liver cells and is a liver damage biomarker that leaks into the blood when liver damage occurs. As shown in Table 11 above, it was confirmed that the inhibitory effect of aspartate aminotransferase (AST), i.e., the liver cell protection effect, in the serum of non-alcoholic steatohepatitis mouse models more increased in the groups administered with a composite composition of the Garcinia extract and Chiisanogenin, as compared to the groups administered with each of them alone.

TABLE 12
Changes of ALT in the serum of non-alcoholic
steatohepatitis (NASH) animal models

| | | | ALT in serum (% | |
|---|---|---|---|---|
| | | | relative to | |
| | | Concen- | positive control | Inhibition rate |
| | | tration | group, mean ± | (%, mean ± |
| No. | Sample | (mg/kg) | deviation) | deviation) |
| 1 | Control material | 0 | 9 ± 1.3 | — |
| | (normal animal) | | | |
| 2 | Control material | 0 | 100 ± 32.0 ## | — |
| | (NASH animal) | | | |
| 3 | Garcinia extract | 200 | 86 ± 18.9 | 15 ± 7.3 |
| 5 | Chiisanogenin | 50 | 89 ± 14.6 | 12 ± 5.1 |
| 6 | Garcinia extract | 75 | 70 ± 9.6 ** | 33 ± 21.7 |
| | Chiisanogenin | 25 | | |
| 7 | Garcinia extract | 150 | 53 ± 13.1 ** | 51 ± 35.9 |
| | Chiisanogenin | 50 | | |

ALT is an enzyme present in liver cells and is a liver damage biomarker that leaks into the blood when liver damage occurs. As shown in Table 12 above, it was confirmed that the inhibitory effect of alanine aminotransferase (ALT), i.e., the liver cell protection effect, in the serum of non-alcoholic steatohepatitis mouse models more increased in the groups administered with a composite composition of the Garcinia extract and Chiisanogenin, as compared to the groups administered with each of them alone.

TABLE 13
Changes of fibrosis marker (hydroxyproline) in liver tissues
of non-alcoholic steatohepatitis (NASH) animal models

| | | | Hydroxyproline in | |
|---|---|---|---|---|
| | | | liver tissues (% | |
| | | Concen- | relative to positive | Inhibition rate |
| | | tration | control group, | (%, mean ± |
| No. | Sample | (mg/kg) | mean ± deviation) | deviation) |
| 1 | Control material | 0 | 45 ± 26 | — |
| | (normal animal) | | | |
| 2 | Control material | 0 | 100 ± 37 ## | — |
| | (NASH animal) | | | |
| 3 | Garcinia extract | 200 | 67 ± 34 | 28 ± 18.2 |
| 5 | Chiisanogenin | 50 | 69 ± 28 | 26 ± 11.9 |
| 6 | Garcinia extract | 75 | 53 ± 36 * | 47 ± 1.2 |
| | Chiisanogenin | 25 | | |
| 7 | Garcinia extract | 150 | 51 ± 18 ** | 50 ± 25.4 |
| | Chiisanogenin | 50 | | |

Hydroxyproline is a main component of collagen and a biomarker of liver fibrosis. As shown in Table 13 above, the hydroxyproline inhibitory effect in the liver tissues of non-alcoholic steatohepatitis animal models, i.e., the liver cell protection effect, more increased in the groups administered with a composite composition of the Garcinia extract and Chiisanogenin, as compared to the groups administered with each of them alone.

As shown in Tables 11 to 13 above, the effect of ameliorating indicators related to non-alcoholic steatohepatitis (NASH) more increased in the groups administered with a composite composition of the Garcinia extract and Chiisanogenin, as compared to the groups administered with each of them alone.

As described above, it was confirmed that the groups administered with a composite composition of the Garcinia extract and Acanthopanax extract or Chiisanogenin showed a significantly improved effect of preventing or treating liver diseases, as compared to the groups administered with each of them alone.

### INDUSTRIAL APPLICABILITY

The composition provided by the present invention can exhibit an increased effect of presenting or treating liver diseases by administering a Garcinia cambogia extract; and an Acanthopanax extract or Chiisanogenin in combination, as compared to administration of each substance alone, and be effective in reducing the possibility of any side effects that may be caused from excessive or long-term administration of each substance. As such, the composition has high industrial applicability.

1. A composition for prevention or treatment of liver diseases, the composition comprising, as active ingredients, (i) a Garcinia cambogia extract, hydroxycitric acid (HCA) or a derivative of hydroxycitric acid; and (ii) an Acanthopanax extract, Chiisanogenin or a derivative of Chiisanogenin.

2. The composition of claim 1, wherein the Garcinia extract and Acanthopanax extract are each independently an extract from a flower, leaf, root, fruit, stem, root bark, stem bark or a combination thereof.

3. The composition of claim 1, wherein the Acanthopanax extract is at least one extract selected from the group consisting of Acanthopanax sessiliflorus (RUPR. et MAX.) SEEM., Acanthopanax senticosus (RUPR. et MAX.) HARMS, Acanthopanax senticosus var. subinermis KITAGAWA, Jiri Mountain Acanthopanax chiisanense NAKAI, Acanthopanax koreanum NAKAI, Acanthopanax rufinerve NAKAI, Acanthopanax seoulense NAKAI, Acanthopanax sieboloians and Acanthopanax sieboldianum MAKINO.

4. The composition of claim 1, wherein the liver disease is selected from the group consisting of non-alcoholic fatty liver disease, non-alcoholic steatohepatitis, alcoholic fatty liver disease, alcoholic liver damage, hepatitis, liver fibrosis, liver cirrhosis, liver failure and liver cancer.

5. The composition of claim 1, wherein the Garcinia cambogia extract and Acanthopanax extract are each independently or simultaneously extracted through (a) solvent extraction using an extraction solvent selected from the group consisting of water, anhydrous or hydrous alcohol having 1 to 4 carbon atoms, propylene glycol, butylene glycol, glycerin, acetone, ethyl acetate, chloroform, butyl acetate, diethyl ether, dichloromethane, hexane and a combination thereof; (b) supercritical extraction; or (c) ultrasonic extraction.

6. The composition of claim 1, wherein the Chiisanogenin is isolated from Acanthopanax.

7-13. (canceled)

14. A method of treating liver diseases, the method comprising administering to a subject in need thereof an effective amount of the composition according to claim 1.

15. The method of claim 14, wherein the liver disease is selected from the group consisting of non-alcoholic fatty liver disease, non-alcoholic steatohepatitis, alcoholic fatty liver disease, alcoholic liver damage, hepatitis, liver fibrosis, liver cirrhosis, liver failure and liver cancer.

16. The method of claim 14, wherein (i) and (ii) are in the form of a single composition or separate compositions.

17. The method of claim 14, wherein (i) and (ii) are administered simultaneously, separately or sequentially.


SEQUENCE LISTING (2 sequences)

SEQ ID NO: 1 - DNA, 22 bp, Artificial Sequence
  Forward Primer
ctgagatcccagcacttcttga

SEQ ID NO: 2 - DNA, 19 bp, Artificial Sequence
  Reverse Primer
gcctccgaagccaaatgag